(19) United States
(12) Patent Application Publication
(10) Pub. No.: US 2023/0158239 A1
(43) Pub. Date: May 25, 2023

(54) Pre-Filled Syringe System and Method

(71) Applicant: Bayer HealthCare LLC, Whippany, NJ (US)

(72) Inventor: Christopher Stout, Pleasanton, CA (US)

(21) Appl. No.: 17/916,602

(22) PCT Filed: Mar. 31, 2021
(86) PCT No.: PCT/US2021/025057

§ 371 Date: Oct. 3, 2022

Related U.S. Application Data
(60) Provisional application No. 63/010,232, filed on Apr. 15, 2020.

(51) Int. Cl. A61M 5/28; A61M 5/46; A61M 5/31

(52) U.S. Cl. CPC A61M 5/28; A61M 5/46; A61M 5/3137

(57) ABSTRACT

This disclosure relates to pre-filled syringes and their methods for use and injection. The embodiments provide a pre-filled syringe system for controlled depth of injection in a subject, including a pre-filled syringe having a needle tip and a plunger for injecting the contents of the pre-filled syringe; a housing for holding the pre-filled syringe and needle tip in a defined position for a depth of injection; and a needle shield mounted on the housing for releasable movement from a locked injection position to a shield position, wherein after the needle shield is moved into the locked injection position with the needle tip extended out of the needle shield and pre-filled syringe system, the needle can be inserted into the subject at a defined depth of injection. The embodiments further provide methods of injecting a subject using a pre-filled syringe system, including providing a pre-filled syringe system having a housing, a needle shield mounted on the housing, and a pre-filled syringe with a needle tip; moving the needle shield mounted on the housing into a locked injection position to extend the needle lip of the pre-filled syringe and system at a defined length out of the needle shield and prc-filled syringe system; inserting the needle tip of the pre-filled syringe and system into a subject at a defined injection depth, and injecting the contents of the pre-filled syringe and system into the subject.

### CROSS-REFERENCE TO RELATED APPLICATIONS

This application claims the benefit of U.S. Provisional Patent Application No. 63/010,232 filed on Apr. 15, 2020, the disclosure of which are hereby incorporated by reference in its entirety.

### BACKGROUND

The pharmaceutical industry uses various devices and methods for delivering drugs. Pre-filled syringes are a way to quickly and easily deliver a drug directly to the blood stream with maximum efficacy. However, subcutaneous injection using a pre-filled syringe does have its challenges with both devices and methods of injection. For instance, self-injection using a pre-filed syringe requires a user to find and maintain a forty-five-degree angle while inserting the needle through their own skin. In addition, in many instances it also requires the user to maintain a skin fold while they inject themselves. This can be difficult to do correctly and if a wrong angle is obtained the needle could penetrate too deeply. Further, in many pre-filled syringe embodiments there is no possible way for controlling the depth in which the needle is inserted. This can allow the needle to penetrate too deep causing, pain, damage, and exposure to skin or blood complications. Additionally, many pre-filled syringe devices in the art do not provide a needle protection to help eliminate needle sticks after the needle injection is complete. Needle stick injuries post injection are a problem for the pharmaceutical industry.

The pre-filled syringes that exist in the art also have challenges for their uses. For instance, in almost all known devices the user must first insert the needle with their thumb and index finger against the barrel of the syringe while also squeezing the barrel while they insert the needle. Next, to perform the injection, the hand must be repositioned and re-oriented while the needle is inside the patient to do the final injection. In some instances, incorrect injections necessitate repeat attempts in order to make the final injection. The device and methods, therefore, provide many challenges to make a correct and effective injection.

Therefore, with these and other challenges in the industry, there is an ongoing need for a pre-filled syringe and method which do not require an injection by the patient at a forty-five-degree angle.

There is still a further need for a pre-filled syringe, system and method which controls the depth of the needle insertion and injection.

Further, there is a need for a pre-filled syringe and system that doesn't require the patient to reposition the needle while the needle is stuck in them.

Still further, there is a need for a pre-filled syringe and system where the syringe provides needle protection after the injection has been completed.

These and other problems experienced in the art have been addressed and obviated by the present embodiments.

### SUMMARY

The embodiments comprise pre-filled syringe devices and their methods of use.

The embodiments provide a pre-filled syringe system for controlled depth of injection in a subject, comprising a pre-filled syringe having a needle tip and a plunger for injecting the contents of the pre-filled syringe; a housing for holding the pre-filled syringe and needle tip in a defined position for a controlled depth of injection; and a needle shield mounted on the housing for movement from a locked injection position to a shield position, wherein after the needle shield is moved into the locked injection position with the needle tip extended out of the needle shield and pre-filled syringe system, the needle can be inserted into the subject at a controlled depth of injection.

The embodiments further provide methods of using and injecting a subject using a pre-filled syringe and system, comprising, providing a pre-filled syringe system having a housing, a needle shield mounted on the housing, and a pre-filled syringe with a needle tip; moving the needle shield mounted on the housing into a locked injection position to extend the needle tip of the pre-filled syringe and system at a defined length out of the needle shield and pre-filled syringe system; inserting the needle tip of the pre-filled syringe and system into a subject at a controlled injection depth, and injecting the contents of the pre-filled syringe and system into the subject.

### BRIEF DESCRIPTION OF THE DRAWINGS

The skilled artisan will understand that the drawings, described below, are for illustration purposes only. The drawings are not intended to limit the scope of the present teachings or claims in any way.

FIG. 1—This figure shows a general pre-filled syringe system.

FIG. 2—This figure shows an exploded view of the pre-filled syringe system and how the components are assembled.

FIG. 3—This figure shows a side elevation view of the needle shield.

FIG. 4A—This figure shows a view of a first end of the needle shield.

FIG. 4B—This figure shows a view of a second end of the needle shield.

FIG. 5—This figure shows a side elevation view of the housing.

FIG. 6A—This figure shows the housing and needle shield in a partial assembled releasable locked injection position without the pre-filled syringe.

FIG. 6B—This figure shows the housing and needle shield in a partial assembled and extended shield position without the pre-filled syringe.

FIG. 7A—This figure shows a cross-sectional view of the pre-filled syringe system and needle tip in a locked injection position.

FIG. 7B—This figure shows a similar cross-sectional view to 7A, but the pre-filled syringe system and needle tip are in a shield position.

FIG. 8A—This figure shows the locking mechanism of the pre-filled syringe system with seated syringe barrel flange prior to rotation into the locked position.

FIG. 8B—This figure shows the locking mechanism of the pre-filled syringe system with seated syringe barrel flange after it is rotated into the locked position.

FIG. 8C—This figure shows a cross-sectional view of the locking mechanism of the pre-filled syringe system.

FIG. 9—This figure shows the method of injection of the pre-filled syringe system including system preparation 9A, injection of subject 9B, system and needle removal 9C, and system and needle tip placed in shield position 9D.

### DETAILED DESCRIPTION

This disclosure describes embodiments related to a pre-filled syringes and methods of using them.

### Definitions

For the purpose of interpreting this specification, the following definitions will apply. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used).

Whenever appropriate, terms used in the singular will also include the plural and vice versa. The use of “a” herein means “one or more” unless stated otherwise or where the use of “one or more” is clearly inappropriate.

The use of “or” means “and/or” unless stated otherwise. The use of “comprise,” “comprises,” “comprising,” “include,” “includes,” and “including” are interchangeable and are not limiting. The terms “such as,” “for example,” and “e.g.” also are not intended to be limiting. For example, the term “including” shall mean “including, but not limited to.”

As used herein, the term “about” refers to +/−10% of the unit value provided.

As used herein, the term “substantially” refers to the qualitative condition of exhibiting a total or approximate degree of a characteristic or property of interest. One of ordinary skill in the biological arts will understand that biological and chemical phenomena rarely, if ever, achieve or avoid an absolute result because of the many variables that affect testing, production, and storage of biological and chemical compositions and materials, and because of the inherent error in the instruments and equipment used in the testing, production, and storage of biological and chemical compositions and materials. The term “substantially” is, therefore, used herein to capture the potential lack of completeness inherent in many biological and chemical phenomena.

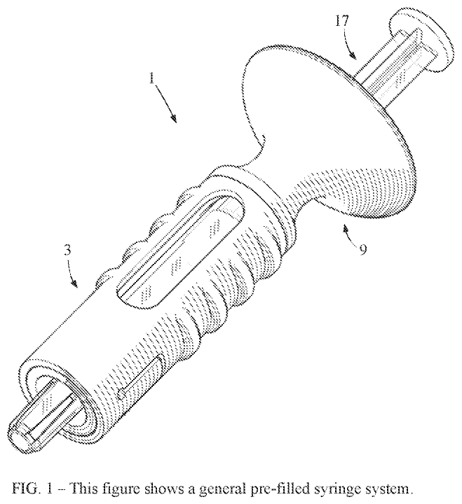

Referring now to the figures, FIG. 1 shows a general perspective view of the pre-filled syringe system 1. The pre-filled syringe system 1, comprises a housing 9, a needle shield 3, and a pre-filled syringe 17. Although the presently described embodiments describe certain components or parts, it is also possible to design the pre-filled syringe system 1 with various other components, orientations, or combinations of elements or parts. Further, each of the components can be further designed to fit in and around each other in various orientations and designs.

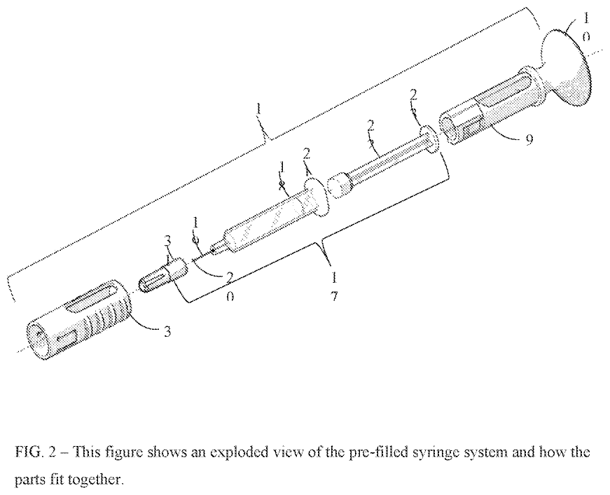

FIG. 2 shows an exploded view of one embodiment of the present invention and how the parts fit together to define the pre-filled syringe system 1. Other embodiments, orientations, and ordering of the parts and assembly can also be possible. As can be seen from FIG. 2 the housing 9 holds the pre-filled syringe 17, and needle shield 3 is then mounted on the housing 9 over the pre-filled syringe 17. The pre-filled syringe 17 can be obtained over the counter or designed for the pre-filled syringe system 1. Various disposable or non-disposable types can be employed with the present embodiments. Pre-filled syringe 17 can comprise a syringe barrel 18 with syringe barrel flange 21, needle 19, needle tip 20, plunger 22 with plunger finger flange 23, and optional needle cap 31. Plunger 22 is designed to fit into syringe barrel 18. Syringe barrel 18 is designed to hold a drug formulation or similar type solution.

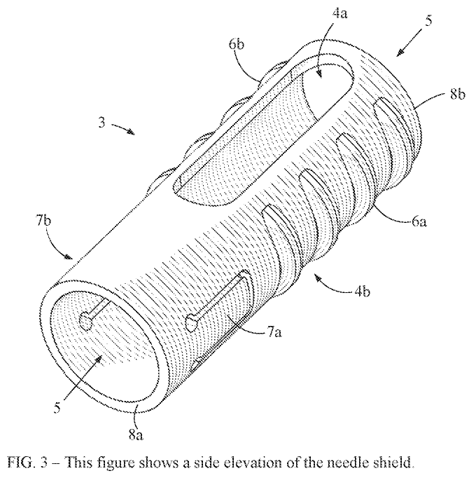

FIG. 3 shows a perspective view of the needle shield 3 of the present embodiments. The needle shield 3 of the present embodiments can be designed to comprise a first needle shield window 4a and a second needle shield window 4b (4b not shown in FIG. 3). The needle shield windows 4a and 4b can be designed so that they are positioned opposite each other. Other orientations and designs are possible. Needle shield 3 can also be designed to comprise a first needle shield finger grip 6a and a second needle shield finger grip 6b as shown in the figures. The first needle shield finger grip 6a and second needle shield finger grip 6b can be designed and positioned opposite each other and orthogonal to the first needle shield window 4a and the second needle shield window 4b. Various types and numbers of finger grips may be employed. Finger grips 6a and 6b are shown in ridge format with two pairs on each side (four on each side or total of 8 ridges). However, other designs are possible. For instance, instead of ridges a series of dimples, notches, or other projection types can be employed for ease of gripping the component.

The needle shield 3 can be configured to further comprise a needle shield elongated bore 5 which passes through the entire component. The needle shield elongated bore 5 can be designed to receive the housing 9 with or without the inserted pre-filled syringe 17. The needle shield 3 can comprise a single part or various different components that fit together.

Referring to FIG. 3, the needle shield 3 of the shown embodiment comprises a first locking tab 7a and a second locking tab 7b. The locking tabs 7a and 7b can be designed to be inwardly facing. The function of the locking tabs 7a and 7b will be further discussed below in conjunction with how they interact with the complementary locking features of the housing 9.

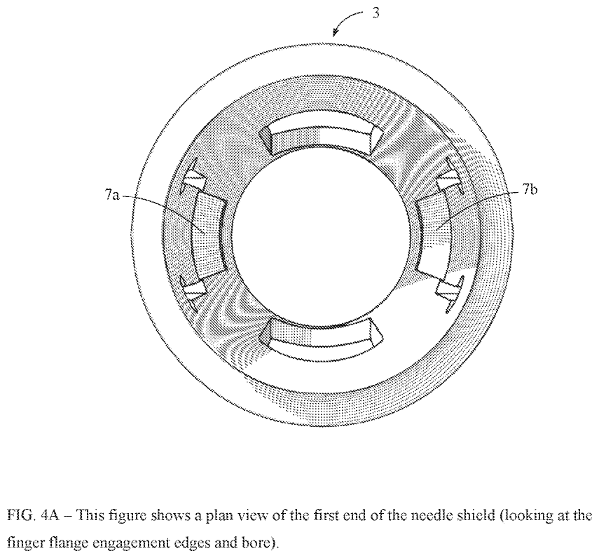

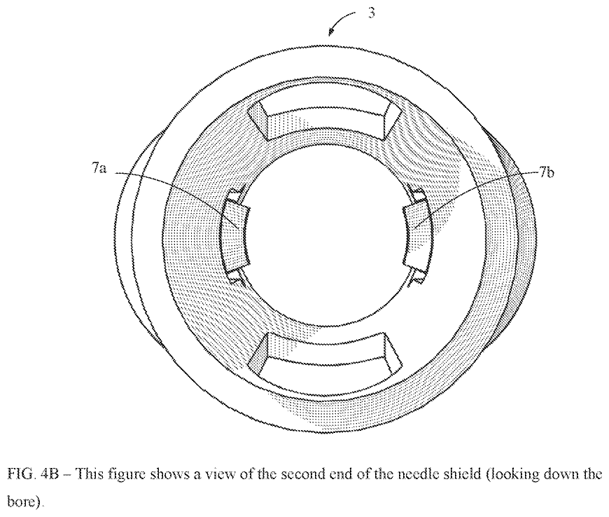

The needle shield 3 can also be designed with various other designs numbers and locking tabs. FIG. 4A shows a plan view of the needle shield 3 from a first end 8a. FIG. 4B shows a plan view of the needle shield 3 from the opposite or second end 8b.

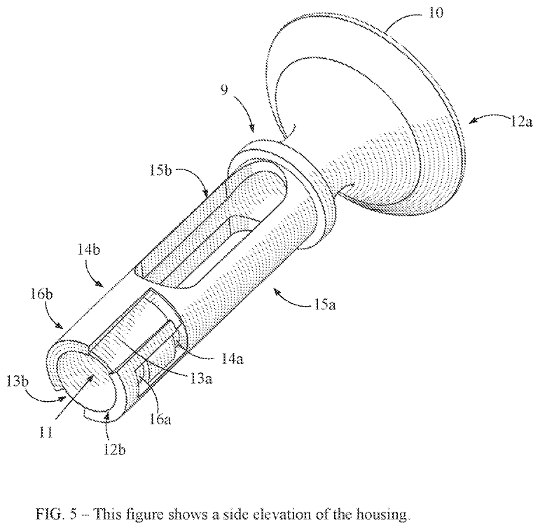

FIG. 5 shows the housing 9 of the present embodiments. The housing 9 can be designed as a single part or a series of components that fit together. The embodiment of housing 9 shown in FIG. 5 comprises a universal finger flange 10. The universal finger flange 10 can be designed in various embodiment sizes and shapes. For purpose of the present embodiments the diameter of the universal finger flange 10 can range from about 5 to about 100 millimeters. The circular and narrow tapering design allows a user to easily grip, hold, and orient the pre-filled syringe system 1 when it is assembled and employed in an injection as will be further discussed below. The universal finger flange 10 as shown in FIG. 5 is designed as part of housing 9. However, it can be imagined in certain embodiments that the universal finger flange 10 can be designed as a separate component with or without a tapered neck and mounted on the housing 9 in various designs and orientations.

Referring again to FIG. 5, the housing 9 can further comprise a housing elongated bore 11. The housing elongated bore 11 is adapted to receive a pre-filled syringe 17 (See FIG. 2 for further details regarding how the parts fit together) when it is assembled and ready for injection of a pharmaceutical drug. The housing elongated bore 11 runs through the entire housing 9 including the finger flange 10. The housing elongated bore 11 receives the pre-filled syringe 17 via the first end 12a near the finger flange 10.

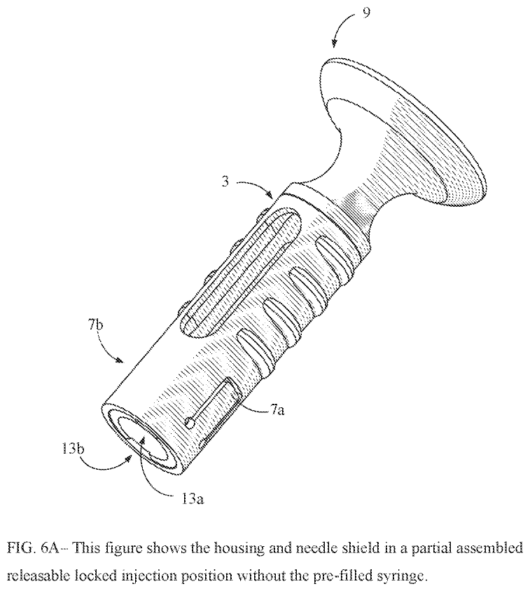

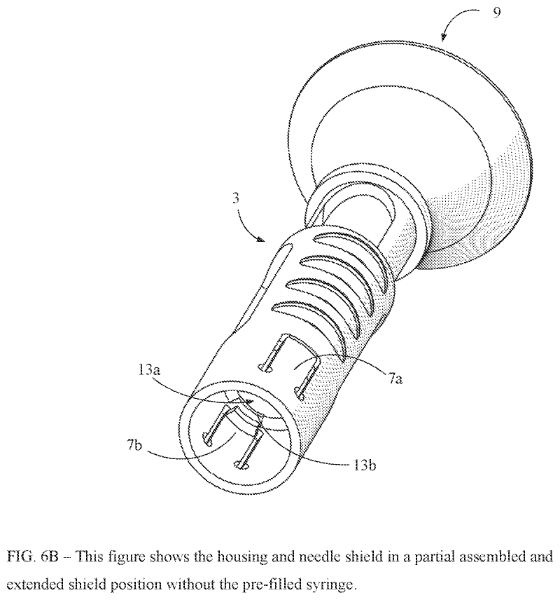

The housing 9 can further comprise a first ramp 13a and second ramp 13b at the second end 12b. The ramps 13a and 13b can be designed to engage the locking tabs 7a and 7b of the needle shield 3 when the components are assembled together. The first ramp 13a and second ramp 13b can be designed to taper inwardly toward the central axis of the housing bore 11. Adjacent to the first ramp 13a and second ramp 13b are first locking notch 14a and second locking notch 14b. Locking notches 14a and 14b are designed to receive and engage the locking tabs 7a and 7b of the needle shield 3. In order for the locking tabs 7a and 7b to engage the locking notches 14a and 14b, the needle shield 3 must first be mounted on the housing 9. FIG. 6A shows the housing 9 and the needle shield 3 in a partial assembled releasable locked injection position without the pre-filled syringe 17. It should be noted that both locking notches 14a and 14b are designed to be releasable. This allows the needle shield 3 to then be later adjusted to slide forward and engage locking notches 16a and 16b as will be further discussed below. FIG. 6B shows the housing 9 and the needle shield 3 in a partial assembled shield position without the pre-filled syringe 17. Locking notches 16a and 16b can preferably be designed to be non-releasable. However, it can be imagined in certain embodiments that they can be designed to be releasable if desired.

Adjacent and spaced near the locking notches 14a and 14b are first locking notch 16a and second locking notch 16b. After the needle shield 3 is mounted on the housing 9 it must be rotated into position so that the locking tabs 7a and 7b drop into position and engage locking notches 14a and 14b. This click-feel engagement allows the user to know when the system is engaged and locked. The pre-filled syringe system 1 is then in position to make an injection while maintaining the needle 19 and pre-filled syringe 17 in a defined locked position and orientation. After the injection is complete, the needle shield 3 can then be released and moved away from the housing 9 to only cover a portion of the housing 9 while shielding the needle 19 and engaging first locking notch 16a and second locking notch 16b.

The housing 9 can further comprise a first window housing 15a and second window housing 15b. The first and second window housings 15a and 15b preferably align with the first and second needle window shields 4a and 4b when the pre-filled syringe system 1 is assembled. This alignment is preferable since it allows the subject 30 to view the level and amount of the formulation in the pre-filled syringe 17 at various desired times. For instance, to determine if the pre-filled syringe is filled prior to injection, is injecting the solution or formulation during an injection, or is empty after an injection.

Referring to FIG. 2, the pre-filled syringe 17 comprises a syringe barrel 18 with syringe barrel flange 21, a needle 19 with a needle tip 20 (with optional cap 31), and a plunger 22 having a plunger finger flange 23. The pre-filled syringe 17 can be designed to hold a pharmaceutical product. Pharmaceutical products can comprise both small molecules and biologics in various formulations. For instance, certain biologics which can be used with the present device can comprise biologics for treatment of hemophilia disease such as Factor VII, Factor VIII, Factor IX, Factor X, Factor XI, insulin, Betaseron®, and other similar small molecules, biologics and their combinations which are known and used in the art. Typically syringe barrel 18 can hold from about 0.25 milliliters of formulation to about 2.0 milliliters of formulation. However, it is within the scope of the present embodiments that various volumes can be employed.

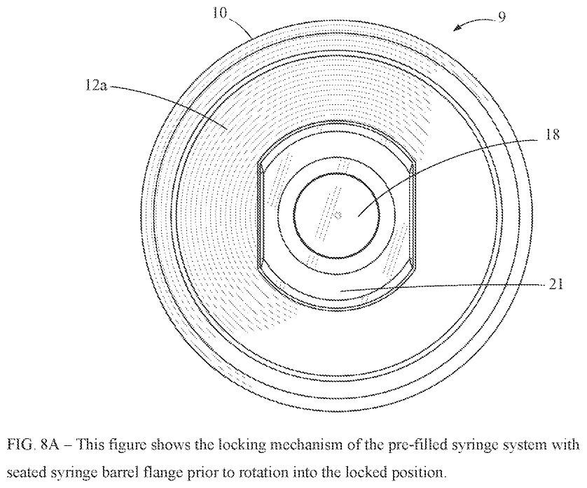

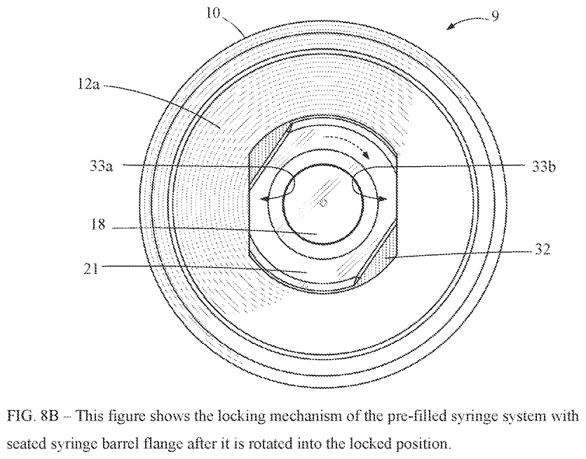

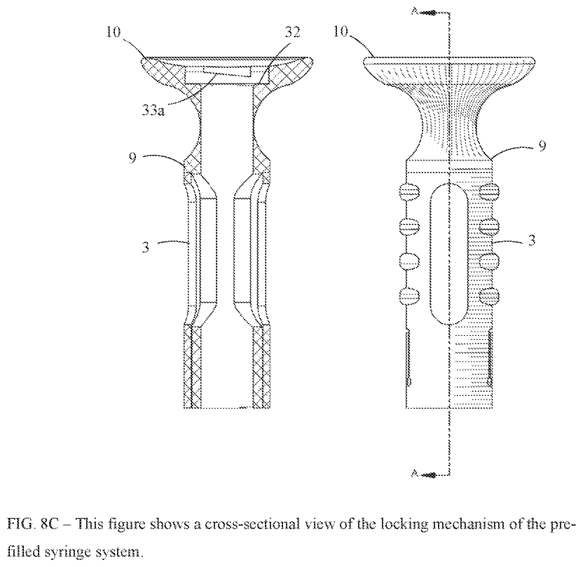

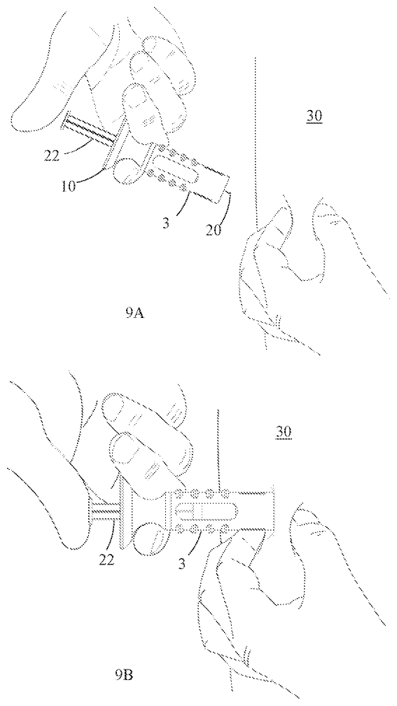

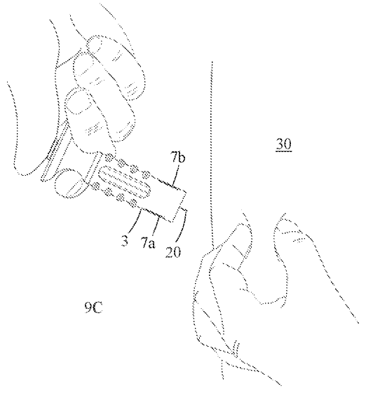

The pre-filled syringe system 1, can further comprise a locking mechanism 33 for attaching the pre-filled syringe 17 to the housing 9 and pre-filled syringe system 1. The locking mechanism 33 can comprise syringe barrel flange 21, mounting surface 32, first locking surface 32a, and second locking surface 32b (See FIG. 8 for details and assembly). It can be imagined that locking mechanism 33 can comprise various parts, organizations, and designs for attaching the pre-filled syringe 17 to the housing 9 and pre-filled syringe system 1. Further, it could be imagined that the locking mechanism 33 could comprise a portion of the pre-filled syringe system 1 (as shown in the present embodiments) or comprise one or more separate parts for attaching the pre-filled syringe 17 onto the housing 9 and pre-filled syringe system 1. Further, barrel flange 21 could be designed in different shapes or sizes for mounting and seating on the first and second locking surfaces 32a and 32b respectively. The first locking surface 32a and the second locking surface 32b could also be designed as a tab or similar type structure.

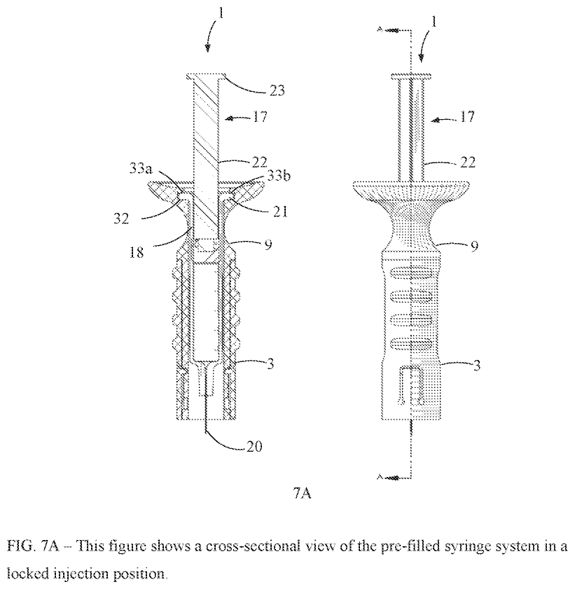

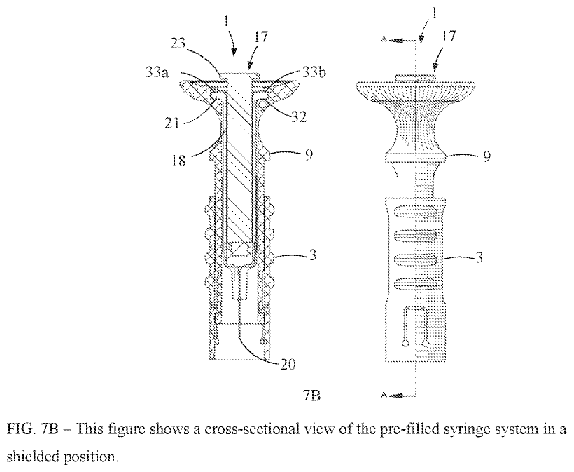

The assembly of the embodiments can begin by placing a pharmaceutical formulation into the syringe barrel 18 of the pre-filled syringe 17. The plunger 22 then is inserted into the syringe barrel 18 behind the pharmaceutical formulation so it contacts or nearly contacts the formulation and any air bubbles are removed from the needle tip 20. Optional cap 31 can be included in the assembly and remain mounted on needle tip 19 to prevent accidental needle sticks. However, optional cap 31 must be removed prior to the injection of the subject 30. The plunger 22 then remains in the loaded, pre-injection position. The pre-filled syringe 17 with formulation is then inserted into the housing bore 11 via the end with the universal finger flange 10 (second end 12a). The pre-filled syringe 17 slides all the way into the housing bore 11 until the syringe barrel 18 and syringe barrel flange 21 are seated against mounting surface 32 of the universal finger flange 10 (See FIGS. 7 and 8). The mounting surface 32 can be designed to be grooved, indented, tapered, dimpled or in other similar type designs or shapes to engage syringe barrel 18 and syringe barrel flange 21 of the pre-filled syringe 17.

The pre-filled syringe 17 should preferably fit tight enough via friction that it does not disengage or fall out of the housing 9. In certain embodiments (as show in the FIGS. 7 and 8) locking mechanism 33 may be employed for attaching the pre-filled syringe 17 to the housing 9 and pre-filled syringe system 1. For instance, after the pre-filled syringe 17 is assembled, mounted in housing 9, and pre-filled syringe flange 21 is seated on mountain surface 32 of the universal finger flange, the locking mechanism 33 can be locked. For instance, the syringe barrel flange 21 can be rotated to engage the first locking surface 32a and the second locking surface 32b to securely lock and attach the pre-filled syringe 17 to the housing 9 and pre-filled syringe system 1 (See FIGS. 8B and 8C). It can be imagined that locking mechanism 33 can be designed in various releasable and non-releasable locking designs and formats.

The plunger 22 of the pre-filled syringe 17 remains in a loaded pre-injection position during this process. Next, the needle shield 3 is mounted on the housing 9 via sliding it over the housing using the needle shield bore 5. The first and second locking tabs 7a and 7b of the needle shield 3 slide along first and second ramps 13a and 13b until they contact the housing 9. The mounting orientation of the needle shield 3 is such that the end having the first and second finger grips 6a and 6b is mounted first on the housing 9. Once the needle shield 3 has been mounted on the housing 9 it is then rotated so that the first and second tabs 7a and 7b drop into the first and second locking notches 14a and 14b. In this orientation, the first and second needle shield windows 4a and 4b are aligned with the first and second housing windows 15a and 15b so that the syringe barrel 18 of the pre-filled syringe 17 with formulation can be viewed. The pre-filled syringe system 1 is now in locked injection mode and ready for use. The user at this point simply needs to remove the needle cap 31 from the pre-filled syringe needle tip 20 and perform the injection. FIGS. 7A and 7B show cross-sectional views of the pre-filled syringe system 1 and pre-filled syringe 17 in the locked and shielded positions and how the needle tip 20 can extend at a defined length from the needle shield 3 and pre-filled syringe system 1 (See FIG. 7A) or be shielded by needle shield 3 (See FIG. 7B).

Having now described the components and their assembly, a description of the method of using the pre-filled syringe system 1 is now in order.

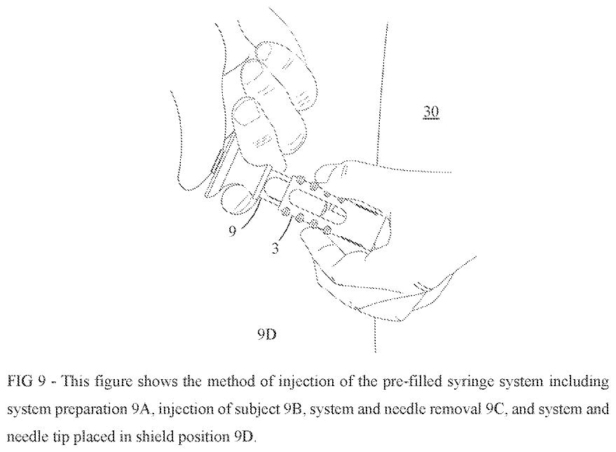

FIG. 9 shows the method of injection of the present embodiments. As previously discussed, the pre-filled syringe system 1 is assembled with a pre-filled syringe 17 having a pharmaceutical formulation. The pre-filled syringe plunger 22 is also in an extended and loaded position once the drug has been loaded. Once the needle shield 3 has been positioned on the housing 9 and placed into the locked position with extended needle tip 20, the device is ready for use. The extent of protrusion of the needle tip 20 can be dependent upon the length of the needle 19, and the size of the needle shield 3. The extension length can be anywhere from 2 to 30 millimeters (as measured from the edge of the needle shield 3) or preferably from about 4 millimeters to about 8 millimeters, depending up the desired depth of injection into the subject.

The subject takes the entire pre-filled syringe system 1 in one hand with fingers around the universal finger flange 10, (FIG. 9A). The needle tip 20 and a portion of the needle 19 is then inserted into the subject 30. Once the needle 19 is inserted it continues until the needle shield 3 contacts the subject 30 and can't penetrate any deeper into the subject's skin (FIG. 9B). Next, the plunger 22 of the pre-filled syringe 17 is depressed and the formulation contents is injected into the subject 30. In the present context, the pharmaceutical drug is delivered into the subject 30 at the desired position and depth. After the injection has been completed the pre-filled syringe system 1 and pre-filled syringe 17 are then removed from the subject 30 (See FIG. 9C). At this point the needle 19 and needle tip 20 are still extended from the needle shield 3 in locked position (as previously described with releasable first locking tab 7a and second locking tab 7b positioned in first locking notch 14a and second locking notch 14b). Pressure is then applied to the sides of needle shield 3 to release first locking tab 7a and second locking tab 7b from the first locking notch 14a and the second locking notch 14b so that needle shield 3 can be slid forward on housing 9 so the first locking tab 7a and second locking tab 7b of the needle shield 3 engage the first locking notch 16a and second locking notch 16b of the housing 9 (See FIG. 9D). As previously discussed, the first and second locking notches 14a and 14b are preferably designed for releasable movement of the needle shield 3 while the first and second locking notches 16a and 16b are not. In this second position, the needle shield 3 now completely shields and encloses the needle 19 and needle tip 20 so they no longer extend outside the pre-filled syringe system 1 to possibly injure the subject 30. The pre-filled syringe system 1 and pre-filled syringe 17 can then be safely disposed of without risk of injuring the subject 30 or others.

We claim:

1. A pre-filled syringe system for controlled depth of injection in a subject, comprising:
(a) a pre-filled syringe having a needle tip and a plunger for injecting the contents of the pre-filled syringe;
(b) a housing for holding the pre-filled syringe and needle tip in a defined position for a depth of injection; and
(c) a needle shield mounted on the housing for releasable movement from a locked injection position to a shield position,
wherein after the needle shield is moved into the locked injection position with the needle tip extended out of the needle shield and pre-filled syringe system, the needle can be inserted into the subject at a defined depth of injection.

2. A pre-filled syringe system as recited in claim 1, wherein the needle tip extends from the needle shield and pre-filled syringe system from about 4 millimeters to about 8 millimeters.

3. A pre-filled syringe system as recited in claim 1, wherein the housing further comprises a universal finger flange.

4. A pre-filled syringe system as recited in claim 1, wherein the housing comprises at least one window for viewing the contents of the pre-filled syringe.

5. A pre-filled syringe system as recited in claim 1, wherein the housing comprises at least one locking notch.

6. A pre-filled syringe system as recited in claim 1, wherein the needle shield comprises one or more finger grips.

7. A pre-filled syringe system as recited in claim 1, wherein the needle shield comprises at least one needle shield window for viewing the injection contents in the pre-filled syringe.

8. A pre-filled syringe system as recited in claim 1, further comprising a locking mechanism for attaching the pre-filled syringe to the housing and the pre-filled syringe system.

9. A method of injecting a subject using a pre-filled syringe system, comprising:
(a) providing a pre-filled syringe system having a housing, a needle shield mounted on the housing, and a pre-filled syringe with a needle tip;
(b) moving the needle shield mounted on the housing into a locked injection position to extend the needle tip of the pre-filled syringe and system at a defined length out of the needle shield and pre-filled syringe system;
(c) inserting the needle tip of the pre-filled syringe and system into a subject at a defined injection depth; and
(d) injecting the contents of the pre-filled syringe and system into the subject.

10. The method of claim 9, wherein the subject can perform the insertion step using one hand.

11. The method of claim 9, wherein the subject can perform the injection step using one hand.

12. The method of claim 9, wherein the needle is inserted into the subject until the needle shield contacts the subject.

13. The method of claim 9, wherein the defined depth of injection is from about 4 millimeters to about 8 millimeters.
